SEQUENCE LISTING (9 sequences)

SEQ ID NO: 1 - DNA, 21 bp, artificial
  RT-PCR primer for CK7
atccgcgagatcaccatcaat

SEQ ID NO: 2 - DNA, 21 bp, artificial
  RT-PCR primer for CK7
atgtgtctgagatctgcgact

SEQ ID NO: 3 - DNA, 37 bp, artificial
  primer to amplify the 5′ arm of the target vector
gtttgagtcgacccaacaaggattacatgagaccgcc

SEQ ID NO: 4 - DNA, 35 bp, artificial
  primer to amplify the 5′ arm of the target vector
taaggcggccgcggatgctcttgcctccttccatg

SEQ ID NO: 5 - DNA, 45 bp, artificial
  primer to amplify the 3′ arm of the target vector
tgcctcgtcgacgcggccgcgctgtgattgcatgtgtgaccttcc

SEQ ID NO: 6 - DNA, 34 bp, artificial
  primer to amplify the 3′ arm of the target vector
caccctcgagttagacctgcagatggtagccgcc

SEQ ID NO: 7 - DNA, 24 bp, artificial
  primer FAHwt-F
aggcctaacctcttgcttcattca

SEQ ID NO: 8 - DNA, 20 bp, artificial
  primer FAHmut-F
ccagctcattcctcccactc

SEQ ID NO: 9 - DNA, 20 bp, artificial
  primer FAHwt/mut-R
atcggggttccagataccac

(19) United States
(12) Patent Application Publication
(10) Pub. No.: US 2009/0275067 A1
(43) Pub. Date: Nov. 5, 2009

(54) METHOD OF ACQUIRING HEPATIC OVAL CELLS

(75) Inventors: Hideki TANIGUCHI, Kobe-shi (JP); Atsushi SUZUKI, Kobe-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi (JP)

(21) Appl. No.: 12/277,371

(22) Filed: Nov. 25, 2008

(30) Foreign Application Priority Data
Feb. 20, 2008 (JP) 2008-039015

(51) Int. Cl. C12Q 1/42; C12Q 1/02; C12N 5/00

(52) U.S. Cl. 435/21; 435/29; 435/325

(57) ABSTRACT

Provided are a method of isolating and identifying hepatic oval cells from the liver, and a method of single cell culture of hepatic oval cells. A method of separating and/or acquiring hepatic oval cells from a mammal, comprising a step for examining the expression of CD133, CD45 and Ter119. Particularly, hepatic oval cells that exhibit the pattern of CD133⁺, CD45⁻ and Ter119⁻ for the three cell surface markers CD133, CD45 and Ter119 are separated and/or acquired, from a mammal. A method of single cell culture of hepatic oval cells, comprising culturing the hepatic oval cells obtained by the above-described method, in the presence of a growth factor and an extracellular matrix.

### FIELD OF THE INVENTION

The present invention relates to a method for separating and acquiring hepatic oval cells. More specifically, the present invention relates to a method of sorting, separating, and acquiring oval cells on the basis of a phenotype characteristic of oval cells.

### BACKGROUND ART

The liver can regenerate in two distinct ways, depending on the cellular compartments undergoing proliferation. After partial hepatectomy (PH) or chemical injury, the cells, in the remaining hepatic tissues, particularly hepatic cells, proliferate rapidly to restore lost cells without any contribution of hepatic stem cells or progenitor cells. However, if hepatic cells proliferation is impaired in some chronic injury, signals for regeneration cause the emergence of small epithelial cells called as “oval cells” around the portal vein in the hepatic lobule. Having the potential for dramatic proliferation to regenerate the damaged liver, and the capability of differentiating into hepatocytes and cholangiocytes, oval cells are thought to be hepatic progenitor cells that proliferate transiently (non-patent document 1).

It has been reported that oval cells can be isolated by centrifugal elutriation and histochemical analysis of various characteristics of parenchymal or non-parenchymal cells of the liver (non-patent document 2). Oval cells are further concentrated by density gradient centrifugation in combination with cell sorting using panning or flow cytometry, and the proliferative capacity and differentiating capacity thereof in vitro or in vivo have been investigated (non-patent documents 3 and 4). Although cell populations isolated by these techniques contained very large numbers of oval cells, they remained mixtures with other lineages of cells; there is a demand for a method of selectively separating oval cells alone.

Although the present inventors and other researchers have reported on strategies for producing hepatic stem cells and progenitor cells from the developing mouse liver (non-patent documents 5 to 7), it has been impossible to analyze hepatic oval cells completely separately from other cells, so that it has been difficult to experimentally demonstrate the pluripotency or tissue regenerative capacity of oval cells.

Non-patent document 1: Fausto N, Campbell J S. The role of hepatocytes and oval cells in liver regeneration and repopulation. Mech Dev 2003; 120: 117-130.
Non-patent document 2: Yaswen P, Hayner N T, Fausto N. Isolation of oval cells by centrifugal elutriation and comparison with other cell types purified from normal and preneoplastic livers. Cancer Res 1984; 44: 324-331.
Non-patent document 3: Germain L, Noel M, Gourdeau H, et al. Promotion of growth and differentiation of rat ductular oval cells in primary culture. Cancer Res 1988; 48: 368-378.
Non-patent document 4: Wang X, Foster M, Al-Dhalimy M, et al. The origin and liver repopulating capacity of murine oval cells. Proc Natl Acad Sci USA 2003; 100 Suppl 1: 11881-11888.
Non-patent document 5: Suzuki A, Zheng Y W, Kondo R, et al. Flow cytometric separation and enrichment of hepatic progenitor cells in the developing mouse liver. Hepatology 2000; 32: 1230-1239.
Non-patent document 6: Suzuki A, Zheng Y W, Kaneko S, et al. Clonal identification and characterization of self-renewing pluripotent stem cells in the developing liver. J Cell Biol 2002; 156: 173-184.
Non-patent document 7: Tanimizu N, Nishikawa M, Saito H, et al. Isolation of hepatoblasts based on the expression of Dlk/Pref-1. J Cell Sci 2003; 116: 1775-1786.

### DISCLOSURE OF THE INVENTION

### Problems to be Solved by the Invention

It is an object of the present invention to provide a method of isolating and identifying hepatic oval cells from the liver. It is another object of the present invention to provide a method of single-cell culture of hepatic oval cells.

### Means of Solving the Problems

In view of the above-described problems, the present inventors conducted extensive investigations, and found that by examining the expression of three kinds of cell surface markers, specifically the expression of the three cell surface markers CD133, CD45 and Ter119, hepatic oval cells can be separated and acquired to high degrees. The inventors established clonal culturing conditions for oval cells, and developed the present invention. Accordingly, the present invention provides the following:

[1] A method of separating and/or acquiring hepatic oval cells from a mammal, comprising a step for examining the expression of CD133, CD45 and Ter119.
[2] The method according to [1] above, further comprising a step for sorting cells that express CD133 and do not express CD45 and Ter119.
[3] The method according to [1] or [2] above, further comprising a step for inducing the production of hepatic oval cells.
[4] The method according to any one of [1] to [3] above, further comprising a step for culturing hepatic oval cells in the presence of a growth factor and an extracellular matrix.
[5] The method according to [4] above, wherein the growth factor is HGF and/or EGF.
[6] The method according to [4] above, wherein the extracellular matrix is collagen or laminin.
[7] Hepatic oval cells separated and/or acquired from the liver of a mammal by the method according to any one of [1] to [6] above.
[8] Hepatic oval cells that exhibit the pattern of CD133⁺, CD45⁻ and Ter119⁻ for the three cell surface markers CD133, CD45 and Ter119.
[9] A method for screening for a substance that influences differentiation in the liver of a mammal, comprising the following steps;
(1) a step for reacting hepatic oval cells and a test substance,
(2) a step for measuring the expression of a liver marker in the cells after the reaction.

### EFFECT OF THE INVENTION

While being the most promising candidate for hepatic stem cells/progenitor cells, oval cells are non-purifiable by any ordinary method of cell separation because of their low abundance, and have been analyzable only in the presence of other cells. For this reason, no experimental evidence has been available so far for identifying oval cells as adult hepatic stem cells/progenitor cells. By the method of the present invention for separating and recovering oval cells with an antigen molecule specifically expressed on the surface of oval cells as an index, and single-cell culture of the oval cells, it is possible to perform a functional analysis of oval cells with extremely high accuracy.

By analyzing oval cell-like cells in human hepatic tissue, particularly in pathologic conditions such as cancer, it is possible to examine their features as cancer stem cells. By using oval cells as hepatic stem cells/progenitor cells, it is possible to establish a screening method for a substance that induces differentiation in the liver.

### BRIEF DESCRIPTION OF THE DRAWINGS

FIG. 1 shows the kinds of primary antibodies used for immunostaining of tissue sections and cultured cells, their availability, reaction conditions and the like.

FIG. 2 shows the procedures of preparing an FAH mutant mouse (null allele). (A) Targeted disruption of the mouse FAH by homologous recombination in ES cells. (B) Southern blotting of parental and targeted ES cell genome using 5′ and 3′ probes. (C) PCR genotyping using genomic DNAs obtained from the tail snips obtained from an FAH+/+ wild-type mouse, an FAH+/− heterozygous mouse, and an FAH−/− mouse (M: marker).

FIG. 3 shows that CD133 expression increases concurrently with the induction of oval cells. (A) Immunohistological staining of A6 in the liver of a No-DDC mouse (upper panel) and the liver of a DDC treated-mouse (lower panel). In 2 weeks of feeding on DDC, oval cell proliferation is induced around the portal vein of the liver. A6 is expressed in both cholangiocytes and oval cells. Scale bar: 500 μm. (B) Multiple gene expression in the livers of No-DDC mice and DDC mice (duration of feeding: 2 weeks) was compared by microarray analysis. This graph shows the data for expression of genes that are increased when oval cell proliferation is activated, as well as for expression of CD133. All data were normalized to the values of the livers of No-DDC mice and fold-differences are shown.

FIG. 4 shows the specific expression of CD133 in cholangiocytes and DDC-induced oval cells. (A) Results of gene expression analysis by qPCR on the livers of No-DDC mice and the livers of mice fed on DDC (1 to 4 weeks). All data were normalized to the values of the livers of No-DDC mice and fold-differences are shown. Each bar shows mean±standard deviation (n=3). (B) Results of Western blotting of protein samples obtained from the livers of No-DDC mice and the livers of mice fed on DDC (1 to 4 weeks). (C-D) Stained images of the livers of No-DDC mice and the livers of mice fed on DDC (2 weeks) by double immunofluorescence staining of CD133 and A6 (FIG. C) or with CD133 and keratin (FIG. D). DNA was stained with DAPI. Scale bar: 100 μm.

FIG. 5 shows the results of in vitro semi-clonal colony analysis of CD133⁺CD45⁻Ter119⁻ cells isolated by flow cytometry. (A) Cells from the livers of No-DDC mice and cells from the livers of DDC mice were fractionated by flow cytometry based on the expression of cell surface markers. First, CD45⁺ cells and Ter119⁺ cells were gated to remove blood system cells from the hepatocytes of the No-DDC mice and the hepatocytes of the DDC mice. CD45⁻Ter119⁻ cells (ratios are shown in each left panel) were then further fractionated on the basis of the expression of CD133. For in vitro colony analysis, a sorting gates were set for the CD133⁺CD45⁻Ter119⁻ cell subpopulation and the CD133⁻CD45⁻Ter119⁻ cell subpopulation. The ratio of CD133⁺ cells in the CD45⁻Ter119⁻ cells and the ratio of CD133⁺ cells in unfractionated total cells are shown in each right panel, shown as percentage ratios outside and inside parenthesis, respectively. Cells from the livers of No-DDC mice, stained with an isotype control antibody, were used as controls (upper panel). (B) For CD133⁺CD45⁻Ter119⁻ cells adhered to glass slides, soon after isolation from the livers of No-DDC mice and the livers of DDC mice by flow cytometry, double immunofluorescence staining of albumin (Alb) and cytokeratin (CK)7 was performed. DNA was stained with DAPI. Scale bar: 50 μm. (C) CD133⁺CD45⁻Ter119⁻ cells isolated from the livers of No-DDC mice and the livers of DDC mice formed semi-clonal colonies after 8 days of culture. Large colonies were formed only from the CD133⁺CD45⁻Ter119⁻ cells obtained from the livers of the DDC mice. The morphologies of the individual colonies are shown. Scale bar: 500 μm (upper panel) and 100 μm (lower panel). (D) For each cell subpopulation derived from the livers of No-DDC mice and the livers of DDC mice, the numbers of small colonies and large colonies per 1000 cells are shown. Each graph shows the mean for 12 dishes (mean±standard deviation) of each cell subpopulation in three independent experiments.

FIG. 6 shows that the colony-forming ability of CD133⁺CD45⁻Ter119⁻ cells derived from the livers of DDC mice is strongly dependent on the growth factor (GFs) and extracellular matrix (ECM) in the culture broth. (A) CD133⁺CD45⁻Ter119⁻ cells were isolated from the liver of a DDC mouse, and cultured in the wells of a 6-well plate coated with type IV collagen, in the presence or absence of a growth factor at a low-density culture condition. The cells could form large colonies after 8 days of cultivation only when HGF and/or EGF was present. Individual colonies are shown. Scale bar: 500 μm (upper panel) and 100 μm (lower panel). (B) A graph showing the numbers of small colonies and large colonies per 1000 cells (CD133⁺CD45⁻Ter119⁻ cells) isolated from the livers of DDC mice. The cells were cultured in a 6-well plate coated with type IV collagen, in the presence or absence of HGF and/or EGF for 8 days to form colonies (n=3, mean±standard deviation). (C) A graph showing the numbers of small colonies and large colonies per 1000 cells (CD133⁺CD45⁻Ter119⁻ cells) isolated from the livers of DDC mice. The cells were cultured in a uncoated 6-well plate (without coating treatment), a 6-well plate coated with type IV collagen, or a laminin-coated 6-well plate, in the presence of HGF and EGF for 8 days to form colonies (n=8, mean±standard deviation).

FIG. 7 shows single cell culture and clonal colony formation of CD133⁺CD45⁻Ter119⁻ cells. (A) Single cell culture of CD133⁺CD45⁻Ter119⁻ cells derived from the liver of a No-DDC mouse. Shown is a representative small colony formed after 8 days of cultivation in the wells of a 96-well plate coated with type IV collagen. (B-C) Magnified views of the small colony shown in (A). Scale bar: 500 μm (B) and 100 μm (C). (D) Single cell culture of CD133⁺CD45⁻Ter119⁻ cells derived from the liver of a DDC mouse. Shown is a representative large colony formed after 8 days of cultivation in the wells of a 96-well plate coated with type IV collagen. (E-F) Magnified views of the large colony shown in (D). Scale bar: 500 μm (E) and 100 μm (F).

FIG. 8 shows multi-lineage colony formation from CD133⁺CD45⁻Ter119⁻ cells derived from the livers of DDC mice (in vitro). (A) For clonal colonies obtained by single cell culture (for 6 days and 18 days) of CD133⁺CD45⁻Ter119⁻ cells isolated from the liver of a No-DDC mouse or the liver of a DDC mouse, double immunofluorescence staining of Alb and CK7 was performed. Representative colonies are shown. DNA was stained with DAPI (lower panel). Scale bar: 100 μm. (B) The ratio of clonal colonies configured with Alb⁺ cells and CK7⁺ cells, Alb⁺ cells and CK7⁻ cells, Alb⁻ cells and CK7⁺ cells, or Alb⁻ cells and CK7⁻ cells, to the colonies formed from a CD133⁺CD45⁻Ter119⁻ cell derived from the liver of a No-DDC mouse or the liver of a DDC mouse, was measured by double immunofluorescence staining of Alb and CK7. The colonies were examined after 6 days and 18 days of cultivation. The data on the small colonies formed from a CD133⁺CD45⁻Ter119⁻ cell derived from the liver of a No-DDC mouse or the liver of a DDC mouse were very similar. This graph shows means for three independent experiments (mean±standard deviation). The number of colonies examined in each experiment was 16 to 62. (C) PAS staining demonstrated that CD133⁺CD45⁻Ter119⁻ cells derived from the liver of a DDC mouse, unlike those from a No-DDC mouse, gave rise to functionally mature hepatocytes with sufficiently accumulated glycogen after 25 days of cultivation. Scale bar: 100 μm. (D) Immunofluorescence staining of Alb for clonal colonies demonstrated that binucleate Alb⁺ mature hepatocytes were differentiated from CD133⁺CD45⁻Ter119⁻ cells from the liver of a mouse fed on DDC after 30 days of cultivation. DNA was stained with DAPI. Scale bar: 100 μm.

FIG. 9 shows the results of an examination of the expression of marker genes of each series for 12 large colonies formed by single cell culture of a CD133⁺CD45⁻Ter119⁻ cell derived from the liver of a DDC mouse. The examination was performed on day 18 using RT-PCR.

FIG. 10 shows the pluripotency of secondary clone-sorted progeny derived from a single CD133⁺CD45⁻Ter119 cell isolated from the liver of a DDC mouse. (A) When the cells were subcultured within each colony for 14 days, secondary colonies were formed from progeny of a single CD133⁺CD45⁻Ter119⁻ cell isolated from the liver of a DDC mouse, whereas no colonies were formed from any progeny of a cell isolated from the liver of a No-DDC mouse. These secondary colonies were stained with Tuerk's solution and photographed. To analyze the self-renewing capacity of CD133⁺CD45⁻Ter119⁻ cells derived from the liver of a DDC mouse, cells in secondary colonies underwent secondary clone-sorting by flow cytometry, and single cell culture was performed. (B) A resorted progeny of a CD133⁺CD45⁻Ter119⁻ cell derived from the liver of a DDC mouse was subjected to single cell culture; after 18 days, double immunofluorescence staining of Alb and CK7 was performed. Representative colonies are shown. DNA was stained with DAPI. Scale bar: 100 μm. (C) The ratio of clonal colonies configured with Alb⁺ cells and CK7⁺ cells, Alb⁺ cells and CK7⁻ cells, Alb⁻ cells and CK7⁺ cells or Alb⁻ cells and CK7⁻ cells, after 18 days of cultivation, was measured by double immunofluorescence staining of Alb and CK7 for these colonies formed from resorted progeny of a CD133⁺CD45⁻Ter119⁻ cell from the liver of a DDC mouse. This graph shows means (mean±standard deviation) for three independent experiments. The number of colonies examined in each experiment is 12 to 21.

FIG. 11 shows that clonogenic progeny propagating in culture from a single CD133⁺CD45⁻Ter119⁻ cell isolated from the liver of a DDC mouse could reconstitute hepatic tissues in vivo. (A) For the livers of an FAH−/− mouse receiving NTBC (upper panel) and an FAH−/− mouse at 2 months after transplantation of cells prepared from culture of clonogenic progeny of a CD133⁺CD45⁻Ter119⁻ cell derived from the liver of a mouse fed on DDC (lower panel), immunohistochemical staining of FAH was performed. Many FAH⁺ hepatic nodules derived from the donor cell were observed in the liver of the recipient mouse. The insets show images of each slides obtained by liver staining with hematoxylin. (B-C) Magnified views of FAH⁺ hepatic nodules derived from donor cells in the liver of a recipient FAH−/− mouse at 1 month (B) or 2 months (C) after transplantation. Scale bar: 100 μm.

FIG. 12 shows hepatocarcinogenesis by p53-deficient CD133⁺CD45⁻Ter119⁻ cells isolated from the liver of a DDC mouse. (A) Despite the lack of p53, clonal large colonies and small colonies were formed from CD133⁺CD45⁻Ter119⁻ cells isolated from the livers of mice fed, or not fed, on DDC. This result is the same as with the CD133⁺CD45⁻Ter119⁻ cells derived from a wild type (p53+/+) mouse. However, the cells proliferating from a CD133⁺CD45⁻Ter119 cell derived from the liver of a p53−/− mouse fed on DDC were smaller than those from a CD133⁺CD45⁻Ter119⁻ cell derived from the liver of a wild type mouse. Scale bar: 100 μm. (B) The clonogenic progeny of a single CD133⁺CD45⁻Ter119⁻ cell isolated from the liver of a p53−/− mouse fed on DDC proliferated more vigorously than the progeny derived from a wild type mouse fed on DDC. 1000 cells were sown to each 6-well plate coated with type IV collagen (n=3, mean±standard deviation). (C-D) A CD133⁺CD45⁻Ter119⁻ cell derived from the liver of a p53−/− mouse fed on DDC was subjected to single cell culture for 18 days, and the large colonies formed were subjected to double immunofluorescence staining of Alb and CK7 (C). Representative colonies are shown. DNA was stained with DAPI (D). Scale bar: 50 μm. (E) The clonogenic progeny of a single CD133⁺CD45⁻Ter119⁻ cell isolated from the liver of a p53−/− mouse fed on DDC formed a tumors at 2 months after subcutaneous injection of cells into a NOD/SCID recipient mouse, whereas the cell isolated from a wild type mouse did not form a tumor. (F) A tumor dissected from the recipient mouse mentioned in (E). Scale bar: 1 cm. (G-H) Hematoxylin-eosin (HE) staining of a tumor region with (H) or without (G) a bile duct-like structure. The data shown in G-K were obtained from the tumor present in F. Scale bar: 100 μm. (I-K) A region of tumor containing many Alb⁺ cells and a few keratin⁺ cells (I), a region of tumor containing many keratin⁺ cells that form a bile duct-like structure and a few Alb⁺ cells (J), and a region of tumor containing both Alb⁺ cells and keratin⁺ cells that form a bile duct-like structure (K). Scale bar: 100 μm.

### BEST MODE FOR CARRYING OUT THE INVENTION

Unless otherwise specified, all technical terms and scientific terms as used herein have the same meanings as those generally understood by those skilled in the technical field to which the invention belongs. Optionally chosen methods and materials that are identical or equivalent to those described herein can be used in embodying or testing the present invention; preferred methods and materials are described below. All publications and patents mentioned herein are incorporated herein by reference, for the purpose of, for example, describing and disclosing any constructions and methodologies described in publications that can be used in relation to the invention described herein.

The hepatic oval cells being subjects of the methods of acquirement and cultivation of the present invention are derived from the liver of an optionally chosen mammal. As mentioned herein, “a mammal” is exemplified by humans, bovines, horses, dogs, guinea pigs, mice, rats and the like. Humans are preferred; from the viewpoint of basic medical research, however, preference is also given to animals in common use for laboratory work, such as mice and rats.

The hepatic oval cells of the present invention are derived from the liver of an optionally chosen mammal, and are preferably derived from a liver treated to induce the production of hepatic oval cells. As an example of the treatment to induce the production of hepatic oval cells, it is possible to produce and proliferate oval cells in the mouse liver by feeding a mouse on a food containing 3,5-diethoxycarbonyl-1,4-dihydrocholidine (DDC) (DDC model). The emergence of oval cells has also been reported in rats undergoing partial hepatectomy after administration of 2-acetylaminofluorene (2-AAF), a kind of carcinogen (2-AAF/PH model).

A liver, preferably of a mouse or the like which is fed on DDC, is removed and shredded, and the cells thereof are dispersed by a physical means such as vibration, or by a chemical treatment with EGTA, EDTA or the like, or by an enzymatic treatment with a protease such as collagenase, trypsin, chymotrypsin, pepsin, or dispase, to obtain a single-cell suspension, after which the step for examining the expression of cell surface markers (CD133, CD45 and TER119) described below is performed.

The present invention provides a method of separating and/or acquiring hepatic oval cells from a mammal, comprising a step for examining the expression of CD133, CD45 and TER119. The CD133 (also known as prominin-1) protein, a glycoprotein with 5 transmembrane domain, is a marker of hematopoietic stem cells and progenitor cells, and has recently been reported to also serve as a cancer stem cell marker of nervous system tumors, prostatic cancer, and colorectal cancer. CD45, a kind of common antigen of leukocytes, is known to be expressed in all hematopoietic cells, except erythrocytes, platelets and progenitor cells thereof. TER119 is known to be a cell surface marker that is effective in sorting erythrocyte-series cells.

The present invention is based on the new finding that these proteins or genes that encode them exhibit unique expression profiles in hepatic oval cells.

As used herein, the term “marker” means each member of the group consisting of a series of proteins that exhibit an expression profile characteristic of such hepatic oval cells, or genes that encode them, unless otherwise specified. As such, marker proteins sometimes have different amino acid sequences depending on the mammalian species and the like; in the present invention, as far as the expression profiles in the hepatic oval cells are the same, such proteins can also be used as marker proteins, falling in the scope of the present invention. Specifically, homologues having an amino acid sequence homology of 40% or more, preferably 50% or more, more preferably 70% or more, of each protein, can also be used as the marker proteins of the present invention, i.e., CD133, CD45 and TER119. The present invention comprises a step for examining the expression of these marker proteins and/or genes that encode them using a substance having specific affinity for each marker protein or a gene that encodes it.

Regarding the term “using” as used herein, the method is not particularly limited. Specifically, for example, when a substance having specific affinity for a marker protein is used, it is possible to use a method based on an antigen-antibody reaction of the marker protein and the antibody. When a substance having specific affinity for a gene that encodes a marker protein is used, it is possible to use a method based on a hybridization reaction (detailed procedures described below).

Substances having specific affinity for a marker protein include, for example, an antibody having specific affinity for the protein or a fragment thereof, the specific affinity being the capability of specifically recognizing, and binding to, the protein by an antigen-antibody reaction. The antibody or a fragment thereof is not particularly limited, as far as it is capable of specifically binding to the protein, and it may be any one of polyclonal antibodies, monoclonal antibodies and functional fragments thereof. These antibodies or functional fragments thereof are prepared by a method in common use in the art. For example, when a polyclonal antibody is used, it is possible to use a method wherein an animal such as a mouse or rabbit is immunized by injecting the protein back-subcutaneously or intraperitoneally or intravenously or otherwise, and an anti-serum is collected after a rise of the antibody titer. When a monoclonal antibody is used, it is possible to use a method wherein a hybridoma is prepared by a conventional method, and the secretion therefrom is collected. A commonly used method of antibody fragment production is to allow a microorganism and the like to express a cloned antibody gene fragment. The purity of the antibody, antibody fragment or the like is not particularly limited, as far as it retains specific affinity for the protein. These antibodies or fragments thereof may be labeled with a fluorescent substance, an enzyme, a radioisotope or the like, with preference given to an antibody labeled with a fluorescent substance, or a fragment thereof.

Furthermore, commercially available supplies may be used.

Examples of a substance having specific affinity for a gene that encodes a marker protein include oligo- or polynucleotide probe (hereinafter also simply referred to as probe for the sake of convenience), and oligo- or polynucleotide primer pair (hereinafter also simply referred to as primer pair for the sake of convenience) which possesses specific affinity for the gene; the specific affinity means the property of hybridizing to the desired gene only; therefore, the substance may be completely complementary to all or part of the gene, or may contain one to several mismatches, as far as the above-described features are ensured. The probe and primer pair are not particularly limited, as long as they have specific affinity for the gene; examples include oligo- or polynucleotide comprising all or part of the base sequence of the gene or a sequence complementary thereto and the like, and they are chosen as appropriate according to the form of the gene to be detected. The oligo- or polynucleotide is not subject to limitations with respect to the derivation thereof, as far as it possesses specific affinity for the gene, and it may be a synthetic product, and may be purified by a conventional method from the necessary portion cleaved out from the gene. These oligo- or polynucleotides may be labeled with a fluorescent substance, an enzyme, a radioisotope or the like.

The method of the present invention for separating and/or acquiring hepatic oval cells is carried out by analyzing the expression of the above-described three markers using respective substances having specific affinity for the three marker proteins or genes that encode them. By separating cells characterized by the pattern of CD133⁺, CD45⁻ and Ter119⁻ [CD133 expressed, CD45 not expressed, TER119 not expressed] by this analysis, hepatic oval cells of mammalian origin can be obtained. Hence, the hepatic oval cells that are to be separated and/or acquired, or to be subjected to single cell culture, in the present invention exhibit the CD133⁺, CD45⁻ and Ter119⁻ pattern for the three cell surface markers CD133, CD45 and Ter119. As used herein, the term “exhibit” means that the property of “expressing” or “not expressing” each marker protein or gene that encodes the same is possessed.

Selection and separation of cells that exhibit the respective marker expression patterns using each substance having specific affinity are normally achieved by a method in use in the art and a combination of such methods. For example, when a marker is analyzed at the protein level, particularly when the cells need to be recovered while being alive, it is advantageous to use a method based on flow cytometry wherein a dye to label the substance is chosen as appropriate. More suitably, the cells are separated using a fluorescence-activated cell sorter (FACS). By using the apparatus, the desired cells can be automatically separated and recovered.

When recovery of viable cells is unnecessary, such as in the case of identification, cells may be disrupted and extracted to recover mRNA, which is subjected to Northern blotting, and membrane protein may be extracted and subjected to Western blotting.

In the present invention, mammalian hepatic oval cells can be obtained using the method of the present invention for separating and/or acquiring hepatic oval cells. Such oval cells permit single cell culture under appropriate culture conditions, and are capable of differentiating into cells with physiological functions, i.e., mature hepatocytes and cholangiocytes. Here, “appropriate culture conditions” mean culture conditions wherein a growth factor and an extracellular matrix are present; examples include culture conditions using a cytokine such as hepatocyte growth factor (HGF) and/or epidermal growth factor (EGF) as the growth factor, and a collagen (e.g., type IV collagen) or laminin as the extracellular matrix. For example, the standard medium described by Suzuki A, Zheng Y W, Kondo R, et al. Flow cytometric separation and enrichment of hepatic progenitor cells in the developing mouse liver. Hepatology 2000; 32: 1230-1239 (non-patent document 5), which comprises HGF and EGF, can be used preferably. The growth factor concentration in the medium is not particularly limited, as far as single cell culture and differentiation induction of hepatic oval cells are possible; varying depending on the kind of growth factor used, the concentration is about 10 to 40 ng/mL, preferably 20 ng/mL, for EGF, and about 40 to 80 ng/mL, preferably 50 ng/mL for HGF. For the growth factors, such as EGF and HGF, used in the present invention, the derivation thereof is not particularly limited, as far as single cell culture and differentiation induction of hepatic oval cells are possible, and they may be of natural derivation, and may be produced by synthesis or semi-synthesis based on publicly known gene sequences, amino acid sequences and other information. Commercially available supplies may be used. As far as single cell culture and differentiation induction of hepatic oval cells are possible, the growth factors, such as EGF and HGF, may be modified ones. Such modifications include mutations, insertion, deletion and substitution of one or several nucleotides or amino acids. A modified growth factor can be prepared by a means in common use in the art. For example, it is possible to artificially modify the DNA of a growth factor by a method of preparing a deletion mutant using an exonuclease, or site-directed mutagenesis techniques such as cassette mutagenesis, and to prepare the desired protein using the modified DNA.

In the present invention, the extracellular matrix is preferably and conveniently used as a coating agent for the culture vessel. Any culture vessel that allows cell culture can be used to culture the cells; examples include flasks, tissue culture flasks, dishes, Petri dishes, tissue culture dishes, multi-dishes, microplates, micro-well plates, multi-plates, multi-well plates, chamber slides, schales, tubes, trays, culturing bags, and roller bottles.

Coating of the culture vessel with an extracellular matrix can be achieved by a method in common use in the art according to the choice of the extracellular matrix used.

Other culture conditions can be set as appropriate. For example, culturing temperature is not particularly limited, and is about 30 to 40° C., preferably about 37° C. CO₂ concentration is, for example, about 1 to 10%, preferably about 2 to 5%.

In the present invention, single cell culture of hepatic oval cells is performed by, for example, sowing cells that exhibit the cell surface marker expression pattern of CD133⁺, CD45⁻ and Ter119⁻, separated from a hepatic cells suspension using a cell separation technique such as FACS, to a 96-well plate or the like at 1 cell per well, and culturing them under appropriate culturing conditions (described above).

Hepatic oval cells, when cultured under appropriate culture conditions, produce differentiated cells, i.e., mature hepatocytes and cholangiocytes, as progeny thereof. Whether or not the cells produced by culturing hepatic oval cells are differentiated cells (mature hepatocytes and cholangiocytes) can be determined by detecting a component specifically expressed in each cell using a substance having specific affinity for the component, or by directly analyzing a secretory component. In some cases, the judgment can be made by morphological examination. For example, whether or not the cells of interest are mature hepatocytes can be determined by examining the presence or absence of albumin production capacity, the presence or absence of glycogen accumulation capacity or the like. Whether or not the cells of interest are cholangiocytes can be determined by examining the presence or absence of a bile duct-like structure, the presence or absence of keratin production capacity or the like. “A substance having specific affinity” includes an antibody having specific affinity for each secretory component or a fragment thereof, an oligo- or polynucleotide probe having specific affinity for the gene that encodes each secretory component. Details of “the antibody or a fragment thereof” and “the oligo- or polynucleotide probe” are as described above.

Regarding mature hepatocytes, because of the glycogen accumulation capacity thereof, the judgment can also be made by a method of staining such as PAS staining or Alcian blue staining.

The present invention further provides a screening method for a substance that influences the differentiation of hepatic oval cells using the hepatic oval cells of the present invention. This method comprises at least the following steps:

(1) A step for reacting hepatic oval cells and a test substance.
(2) A step for measuring the expression of a liver marker in the cells after the reaction.

The hepatic oval cells used in the present screening method are not particularly limited, as far as they differentiate into functional cells of the liver, and they are preferably hepatic oval cells separated by the above-described method of the present invention. Reaction conditions for the oval cells and a test substance are set as appropriate according to the desired type of differentiation, differentiation induction conditions and the like; the cells are normally cultured at 35 to 40° C. for several minutes to several tens of days, preferably at 36.5 to 37.5° C. for 5 to 30 days. After the reaction with the test substance, the presence or absence and status of differentiation of the oval cells are examined.

Whether or not the hepatic oval cells have differentiated can be determined by measuring the expression of a marker of the liver. The marker is chosen as appropriate according to the desired functional cells. For example, as described above, the differentiation can be confirmed by examining albumin production or glycogen accumulation in the case of differentiation into mature hepatocytes, or by examining keratin production and the like in the case of differentiation into cholangiocytes. A substance that significantly promotes or inhibits the differentiation thereof into liver functional cells, compared with hepatic oval cells cultured in the absence of the test substance, is selected.

Test substances that can be subjects of the screening method of the present invention include a wide variety of known or unknown substances; specifically, examples include known cytokines, extracellular matrices, inorganic compounds, or unknown genes obtained from culture supernatants of appropriate cell lines in culture or appropriate cDNA libraries, or recombinant proteins thereof and the like.

Furthermore, because it has been reported that transformed oval cells can be a candidate for the origin of hepatocarcinoma in both rodents and humans (references 1 to 5), it is possible to screen for a substance that influences hepatocarcinogenesis using the hepatic oval cells of the present invention.
Reference 1: Factor V M, Radaeva S A. Oval cells-hepatocytes relationships in Dipin-induced hepatocarcinogenesis in mice. Exp Toxicol Pathol 1993; 45: 239-244.
Reference 2: Lowes K N, Brennan B A, Yeoh G C, et al. Oval cell numbers in human chronic liver diseases are directly related to disease severity. Am J Pathol 1999; 154: 537-541.
Reference 3: Prior P. Long-term cancer risk in alcoholism. Alcohol Alcohol 1988; 23: 163-171.
Reference 4: Tsukuma H, Hiyama T, Tanaka S, et al. Risk factors for hepatocellular carcinoma among patients with chronic liver disease. N Engl J Med 1993; 328: 1797-1801.
Reference 5: Deugnier Y M, Guyader D, Crantock L, et al. Primary hepatocarcinoma in genetic hemochromatosis: a clinical, pathological, and pathogenetic study of 54 cases. Gastroenterology 1993; 104: 228-234.

### EXAMPLES

The present invention is hereinafter described in more detail by means of the following examples, which, however, are not to be construed as limiting the scope of the invention. The reagents, apparatuses, and materials used in the present invention are commercially available unless otherwise specified.

### (Materials and Methods)

### Flow Cytometry

Cell suspensions containing oval cells were prepared from C57BL/6 wild type mice or p53−/− mice reared with a food containing, or not containing 0.1% DDC (Sigma-Aldrich) for 2 weeks (Reference 6); subsequently, the dual-protease digestion protocol shown below was performed. After an ordinary 2-step perfusion process using a liver perfusion medium (Invitrogen) and a liver digest medium (Invitrogen), the undigested tissue was further digested with dispase (1000 protease units/mL; Godoshusei, Japan) while stirring at 37° C. for 30 to 60 minutes. Cell viability after treatment exceeded 90% as determined by an analysis using the trypan blue dye exclusion method. The cells were washed with a staining medium (PBS containing 3% FBS), and then incubated with fluorescence-conjugated antibodies as described in Reference 7. A phycoerythrin (PE)-Cy7-conjugated anti-CD45 and anti-Ter119 monoclonal antibodies (mAbs) (Pharmingen) and a fluorescein isothiocyanate (FITC)-conjugated anti-CD133 monoclonal antibody (eBioscience) were used. The fluoresce-labeled cells were analyzed and separated using FACS Aria (BD Biosciences).
Reference 6: Tsukada T., et al. Enhanced proliferative potential in culture of cells from p53-deficient mice. Oncogene 1993; 8: 3313-3322.
Reference 7: Suzuki A., et al. Clonal identification and characterization of self-renewing pluripotent stem cells in the developing liver. J Cell Biol 2002; 156: 173-184.

### In Vitro Colony Assay

For low-density culture analysis, sorted cells were sown to a 6-well plate coated with type IV collagen (BD Biosciences) at a density of 1000 cells/well, and cultured in the standard medium described in reference 8. For single cell culture analysis, cells identified by clone-sorting by FACS Aria were cultured in the individual wells of a 96-well plate coated with type IV collagen (BD Biosciences). The culture broth used was the standard medium (described above) supplemented with 50% of medium conditioned by 7 days culture of E13.5 embryonic hepatic cells. Both culture broth contained human recombinant HGF (50 ng/ml, Sigma-Aldrich) and EGF (20 ng/ml, Sigma-Aldrich). The large colonies and small colonies formed were counted after 8 days of cultivation.
Reference 8: Suzuki A., et al. Flow cytometric separation and enrichment of hepatic progenitor cells in the developing mouse liver. Hepatology 2000; 32: 1230-1239.

### Gene Expression Analysis

RT-PCR and quantitative PCR (qPCR) were performed as described in references 9 and 10. Various PCR primers and probes were prepared as reported by the present inventors (references 7 to 9) except for the primers and/or probes shown below.

### CK7 RT-PCR Primers:

(SEQ ID NO: 1)

5′-ATC CGC GAG ATC ACC ATC AAT-3′
and

(SEQ ID NO: 2)

5′-ATG TGT CTG AGA TCT GCG ACT-3′

CD133 qPCR Primer/Probe:

TaqMan Gene Expression Assay ID: Mm00477115_ml (Applied Biosystems)

CK7 qPCR Primer/Probe:

TaqMan Gene Expression Assay ID: Mm00466676_ml (Applied Biosystems)
Reference 9: Suzuki A., et al. Role for growth factors and extracellular matrix in controlling differentiation of prospectively isolated hepatic stem cells. Development 2003; 130: 2513-2524.
Reference 10: Suzuki A., et al., Glucagon-like peptide 1 (1-37) converts intestinal epithelial cells into insulin-producing cells. Proc Natl Acad Sci USA 2003; 100: 5034-5039.

### Immunostaining

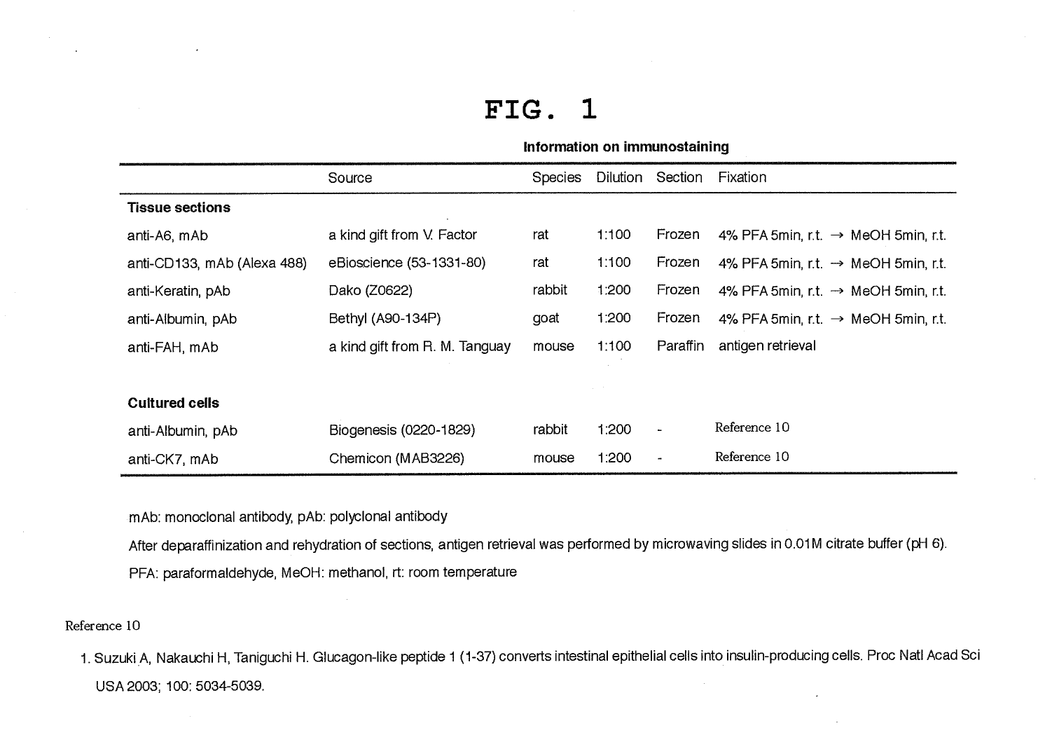

Tissue sections and cultured cells were fixed, and incubated with primary antibodies. The primary antibodies used, the availability thereof, reaction conditions and the like are summarized in FIG. 1. After the incubation with the primary antibodies, the sections and/or cells were washed. For immunohistochemical examination, a horse radish peroxidase (HRP)-conjugated secondary antibody (chosen according to the kind of primary antibody used, 1:500, Dako) was used; for immunofluorescence staining, an Alexa 488- and/or Alexa 555-conjugated secondary antibody (chosen according to the kind of primary antibody used, 1:200, Molecular Probes) and DAPI were used.

### Cell Transplantation

Clonogenic progeny obtained by culturing and proliferating a single cell that exhibits the CD133⁺CD45⁻Ter119⁻ phenotype isolated from a DDC-treated liver were trypsinized, washed, and re-suspended in 100 μl of the standard medium. The resulting cell suspension was administered intrasplenically to the livers of FAH-deficient recipient mice (described below) (4×10⁶ cells/mouse). Twelve clones that exhibit the CD133⁺CD45⁻Ter119⁻ phenotype were established. Three clones thereof were used as donor cells for transplantation (6 recipient mice used per clone). Although FAH-deficient mice were normally reared with drinking water containing 7.5 mg/l NTBC (supplied by K. Z. Travis and J. Doe) (reference 11), this treatment was discontinued immediately after the cell transplantation. For tumorigenesis analysis, clonogenic progeny obtained by culturing and proliferating a single cell that exhibits the CD133⁺CD45⁻Ter119⁻ phenotype isolated from the liver of a wild type mouse or p53−/− mouse fed on a DDC-containing food were trypsinized, washed, and re-suspended in the standard medium (150 μl) containing Matrigel (150 μl, BD Biosciences), and the resulting cell suspension was injected subcutaneously to NOD/SCID recipient mice (3.5×10⁷ cells/mouse, n=5).
Reference 11: Overturf K., et al., Hepatocytes corrected by gene therapy are selected in vivo in a murine model of hereditary tyrosinaemia type I. Nat Genet 1996; 12: 266-273.

### Microarray

Total RNA was prepared from the livers of mice fed on a DDC-free food, or fed on a DDC-containing food for 2 weeks, using the RNeasy Mini Kit (Qiagen), as directed in the manufacturer's instructions. Multiple gene expression in the livers of the mice fed on the DDC-free food, or fed on the DDC-containing food, was analyzed using One-Cycle Target Labeling and Control Reagents (Affymetrix) and GeneChip Mouse Genome 430 2.0 Arrays (Affymetrix), as directed in the GeneChip Expression Analysis Technical Manual (Affymetrix).

### Generation of FAH Mutant Mouse

Targeted disruption of mouse FAH was performed by homologous recombination in TT2 embryonic stem (ES) cells, as described in references 12 to 15. PCR was performed to amplify the 5′-arm (8.2 kb) and 3′-arm (5.1 kb) of the targeting vector sequence. The primers used are as follows:

Primers for 5′-arm:

(SEQ ID NO: 3)

5′-GTT TGA GTC GAC CCA ACA AGG ATT ACA TGA GAC CGC
C-3′

(SEQ ID NO: 4)

5′-TAA GGC GGC CGC GGA TGC TCT TGC CTC CTT CCA TG-
3′
Primers for 3′arm:

(SEQ ID NO: 5)

5′-TGC CTC GTC GAC GCG GCC GCG CTG TGA TTG CAT GTG
TGA CCT TCC-3′

(SEQ ID NO: 6)

5′-CAC CCT CGA GTT AGA CCT GCA GAT GGT AGC CGC C-3′

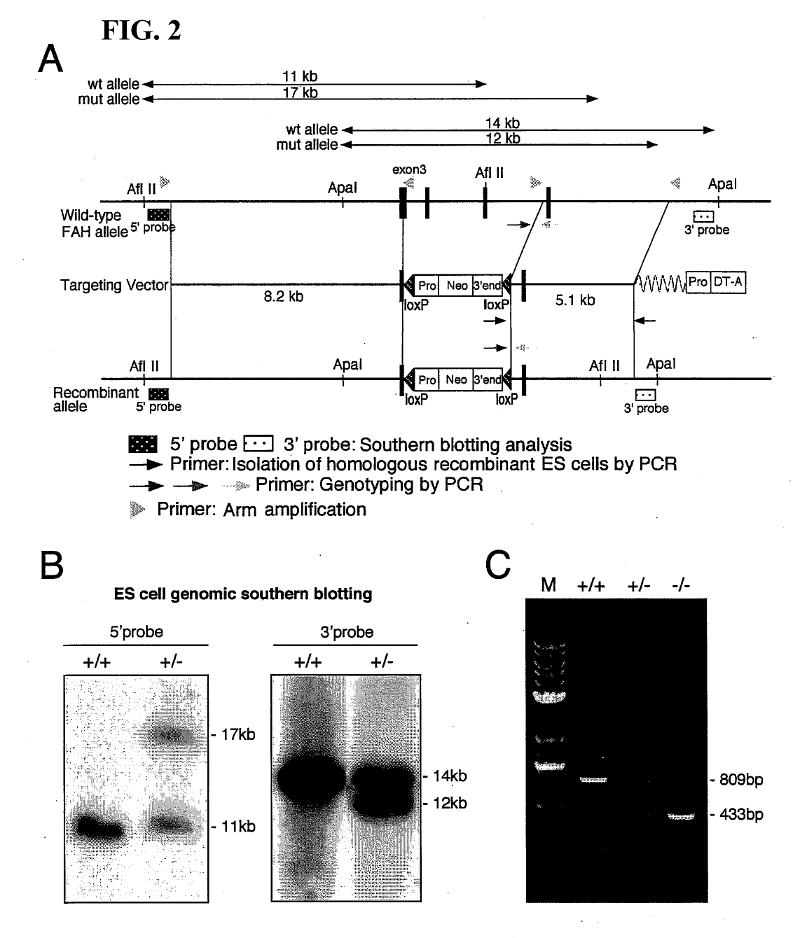

The procedures for generating an FAH mutant mouse (null allele) are shown in FIG. 2A.

Germline chimeras were prepared by injecting targeted ES cells into blastocysts of a CD-1-series host (reference 14). Genotyping was performed by PCR. The primers used are as follows:

FAHwt-F:

(SEQ ID NO: 7)

5′-AGG CCT AAC CTC TTG CTT CAT TCA-3′
FAHmut-F:

(SEQ ID NO: 8)

5′-CCA GCT CAT TCC TCC CAC TC-3′

(SEQ ID NO: 9)

FAHwt/mut-R; 5′-ATC GGG GTT CCA GAT ACC AC-3′

FAHwt-F and FAHwt/mut-R were used for the wild types to detect 809 bp.

FAHmut-F and FAHwt/mut-R were used for the mutant allele to detect 433 bp. (see FIG. 2C)

Note that the accession number (RIKEN) for the FAH mutant is CDB0201K.
Reference 12: Yagi T., et al. Homologous recombination at c-fyn locus of mouse embryonic stem cells with diphtheria toxin A fragment gene in negative selection. Proc Natl Acad Sci USA 1990; 87: 9918-9922.
Reference 13: Yagi T., et al. A novel negative selection for homologous recombinants using diphtheria toxin A fragment gene. Anal Biochem 1993; 214: 77-86.
Reference 14: Yagi T, Tokunaga T, Furuta Y, et al. A novel ES cell line, TT2, with high germline-differentiating potency. Anal Biochem 1993; 214: 70-76.
Reference 15: Murata T., et al. ang is a novel gene expressed in early neuroectoderm, but its null mutant exhibits no obvious phenotype. Gene Expr Patterns 2004; 5: 171-178.

### Example 1

### Expression of CD133 in Cholangiocytes and DDC-Induced Oval Cells

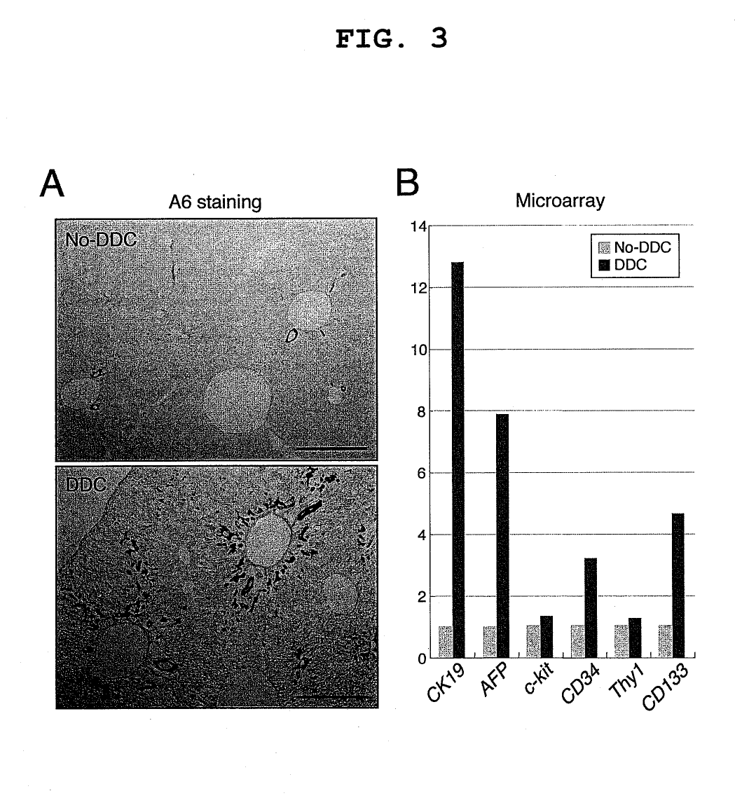

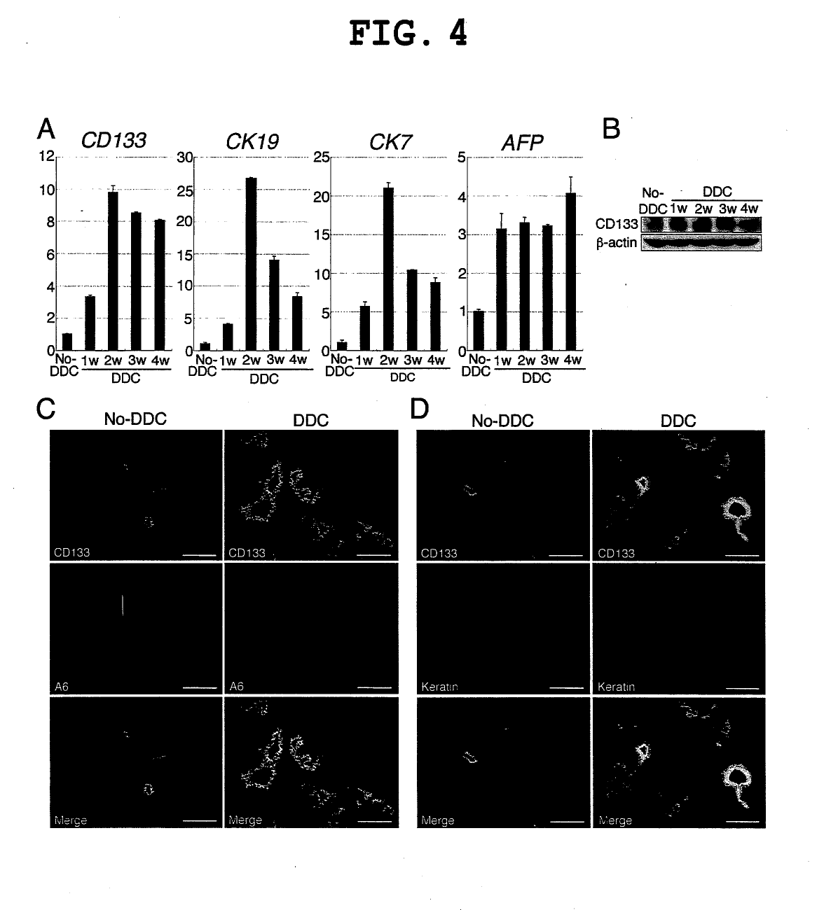

It was confirmed, by immunohistological analysis of A6, a specific antigen for oval cells, that oval cells emerged in the livers of mice fed on DDC for 2 weeks (hereinafter also referred to as DDC mice) (FIG. 3A). To demonstrate the expression of cell surface markers on oval cells, the present inventors compared gene expression in the livers of mice not fed on DDC (hereinafter also referred to as No-DDC mice) and the livers of DDC mice by means of a microarray. This analysis confirmed a higher expression of cytokeratin (CK)19, α-fetoprotein (AFP) and CD34, and to a lesser extent, c-kit and Thy1, in the livers of the DDC mice. These genes each have been previously identified as an oval cell marker (references 16 to 20). In addition to these marker genes, the expression level of CD133 increased 4 fold or more in the livers of the DDC mice (FIG. 3B). Subsequently, quantitative PCR (qPCR) analysis was performed, confirming increased expression levels of CD133, as well as of the oval cell markers CK19, CK7 and AFP, throughout the period of feeding on a DDC-containing food (FIG. 4A). The CD133 protein itself also accumulated in the livers of the DDC mice (FIG. 4B). Interestingly, the expression levels of the mRNAs of CD133, CK19 and CK7 concurrently reached their peaks after 2 weeks of rearing with the DDC-containing food, i.e., feeding on DDC, whereas the AFP level increased rapidly until 1 week after the start of feeding on DDC (FIG. 4A). Analysis by immunofluorescence staining showed that both the cholangiocytes of the livers of the No-DDC mice and the oval cells proliferating in the livers of the DDC mice were positive for CD133 (FIG. 4C to D). These results showed that CD133 could serve as a marker for both the cholangiocytes of the livers of mice not fed on DDC and the oval cells that emerge with feeding on DDC.
Reference 16: Lemire J M, Shiojiri N, Fausto N. Oval cell proliferation and the origin of small hepatocytes in liver injury induced by D-galactosamine. Am J Pathol 1991; 139: 535-552.
Reference 17: Fujio K, Evarts R P, Hu Z, et al. Expression of stem cell factor and its receptor, c-kit, during liver regeneration from putative stem cells in adult rat. Lab Invest 1994; 70: 511-516.
Reference 18: Omori N, Omori M, Evarts R P, et al. Partial cloning of rat CD34 cDNA and expression during stem cell-dependent liver regeneration in the adult rat. Hepatology 1997; 26: 720-727.
Reference 19: Petersen B E, Goff J P, Greenberger J S, et al. Hepatic oval cells express the hematopoietic stem cell marker Thy-1 in the rat. Hepatology 1998; 27: 433-445.
Reference 20: Matsusaka S, Tsujimura T, Toyosaka A, et al. Role of c-kit receptor tyrosine kinase in development of oval cells in the rat 2-acetylaminofluorene/partial hepatectomy model. Hepatology 1999; 29: 670-676.

### Example 2

### Isolation of CD133⁺ Cells Using Flow Cytometry

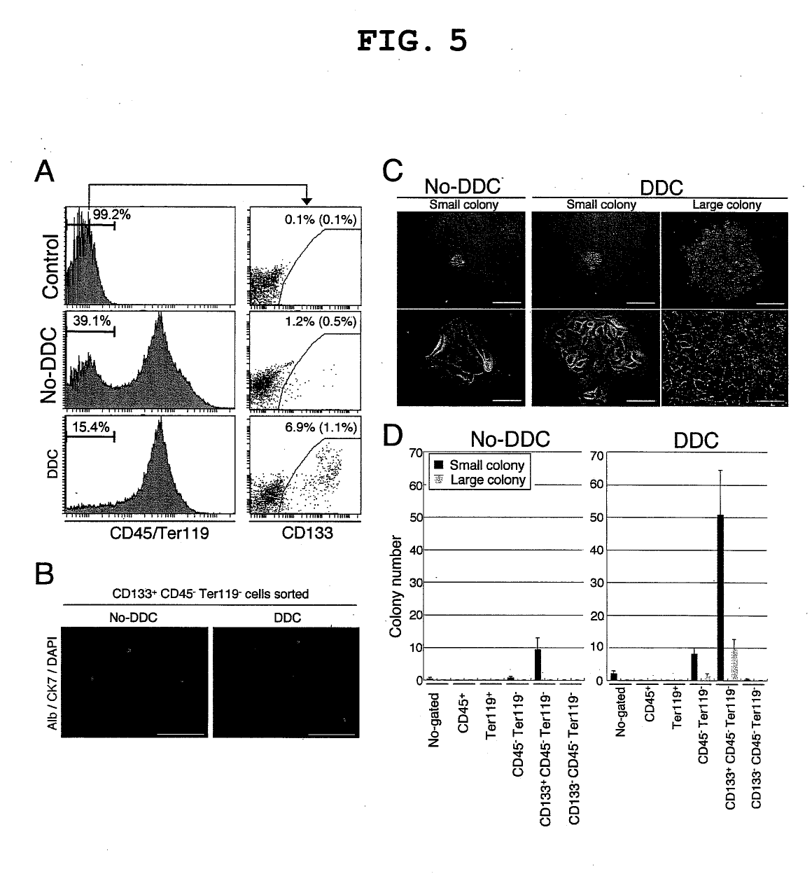

In order to isolate CD133⁺ hepatic cells, single cells obtained from the liver of a No-DDC mouse and the liver of a DDC mouse were fractionated using flow cytometry on the basis of the expression of CD133, CD45 (a common leukocyte antigen) and Ter119 (a molecule resembling glycophorin and exclusively expressing on immature erythroid cells). The ratio of CD133⁺ cells in a non-hematopoietic CD45⁻Ter119⁻ cell population was higher by 5 times or more for the livers of mice fed on DDC than for the livers of mice not fed on DDC (FIG. 5A). The increase in the ratio of CD45⁺ and Ter119⁺ hematopoietic cells in the livers of DDC mice may be related to the enhanced inflammatory response caused by DDC. Immunofluorescence staining analysis of CD133⁺CD45⁻Ter119⁻ cells immediately isolated from the liver of a No-DDC mouse and the liver of a DDC mouse showed the expression of CK7 in all cells with no albumin expressed (albumin is a marker protein of hepatic cell) (FIG. 5B).

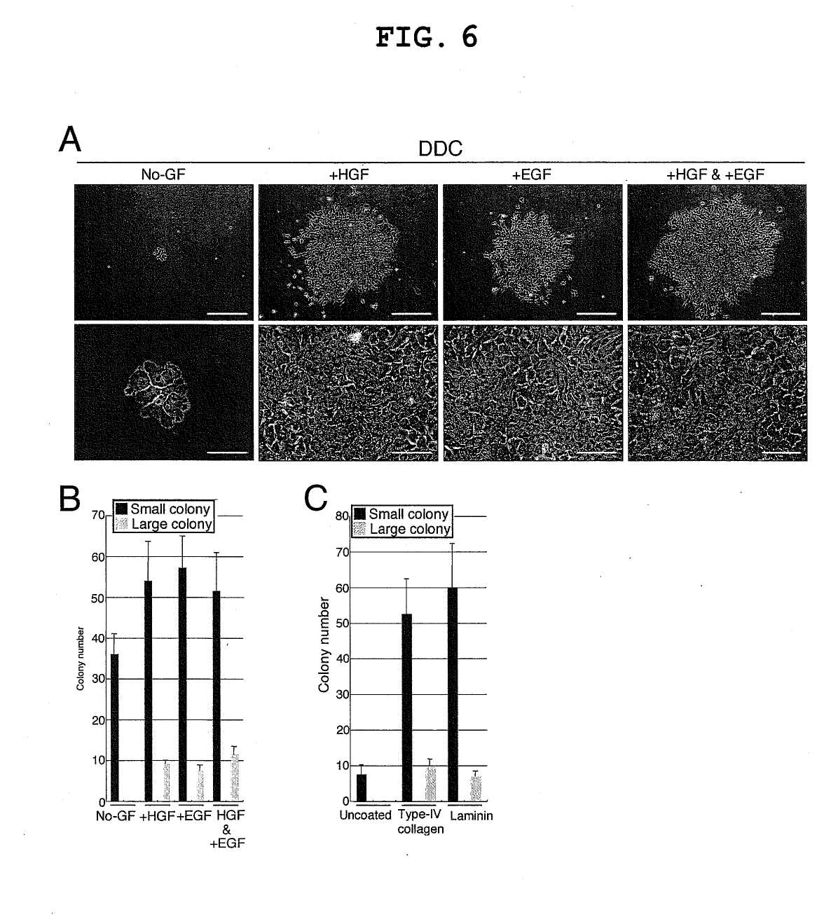

Next, to characterize CD133⁺CD45⁻Ter119⁻ cells isolated from the livers of No-DDC mice and the livers of DDC mice, these cells were cultured at low density. 1000 cells were sown to each well of a 6-well plate coated with type IV collagen. Because 6-well plates allow cell proliferation and colony formation, semi-clonal analysis is possible. Under these conditions, the CD133⁺CD45⁻Ter119⁻ cells isolated from the livers of No-DDC mice only formed small colonies configured with 10 to 50 cells, even after 8 days of cell culture (FIG. 5C). On the other hand, the CD133⁺CD45⁻Ter119⁻ cells isolated from the livers of DDC mice were capable of forming not only small colonies, but also large colonies configured with several hundreds of cells (FIG. 5C). These two types of cell colonies were easily distinguishable from each other by their size during cultivation. When CD133⁺CD45⁻Ter119⁻ cells were isolated and cultured, they frequently produced both small colonies and large colonies; however, the cells isolated from a CD45⁺Ter119⁺ cell subpopulation or CD133⁻CD45⁻Ter119⁻ cell subpopulation did not colonize at all, or colonized to very low extent (FIG. 5D). The emergence of cells that form large colonies was strongly dependent on DDC treatment, and the number of small colonies was also increased 5 times or more in the livers of mice fed on DDC (FIG. 5D). Furthermore, it was found that for the formation of large colonies by CD133⁺CD45⁻Ter119⁻ cells derived from the liver of a DDC mouse, a growth factor [hepatocyte growth factor (HGF) and/or epithelial growth factor (EGF)] and an extracellular matrix (ECM) component (type IV collagen or laminin) are required as culture conditions (see FIG. 6).

### Example 3

### Single Cell Culture and Clonal Analysis of CD133⁺CD45⁻Ter119⁻ Cells

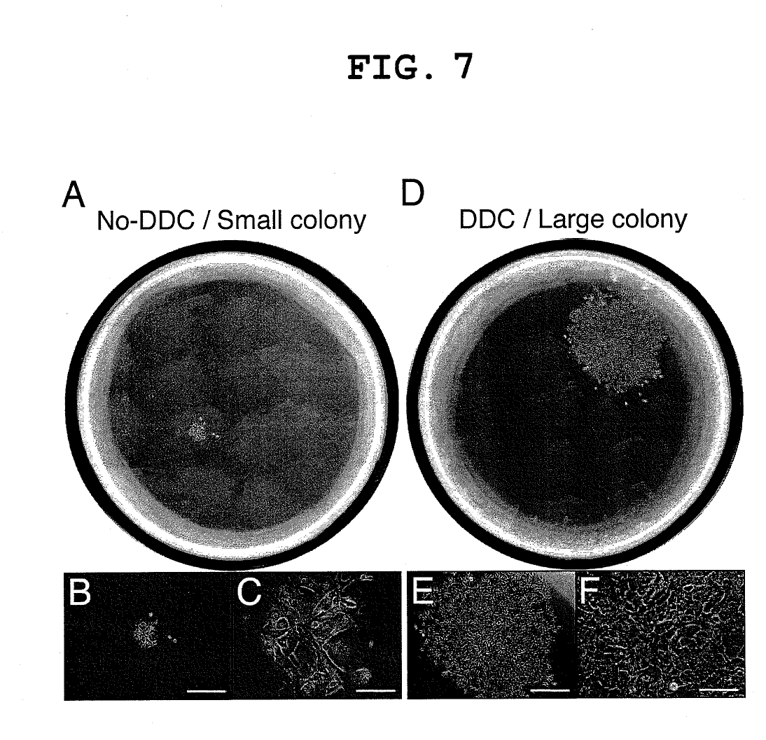

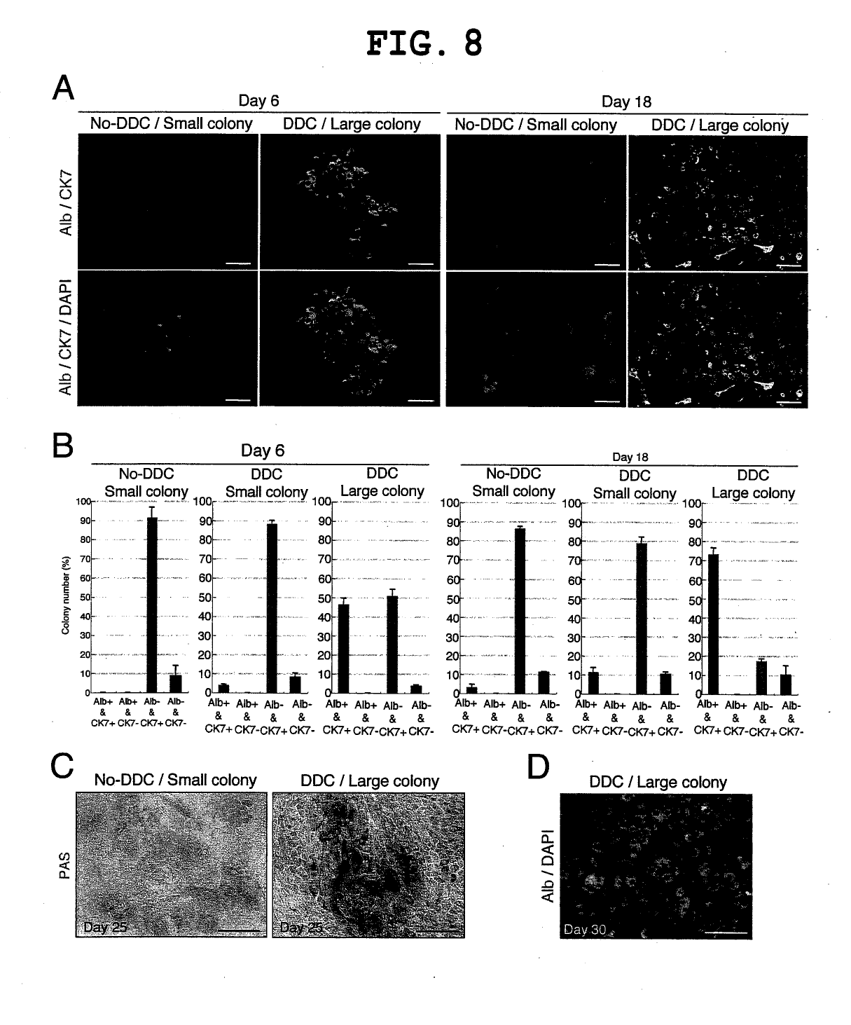

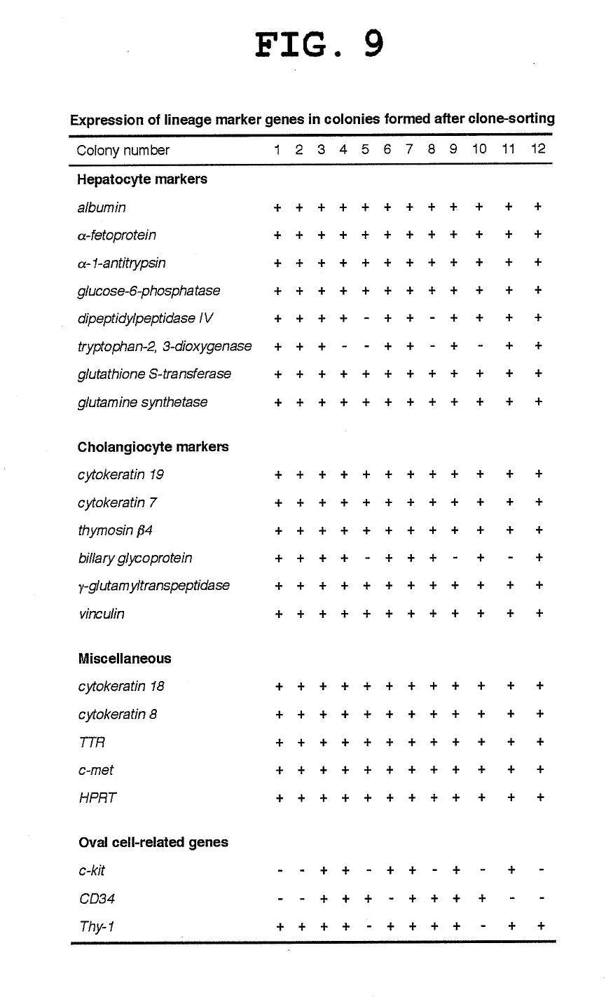

To examine the potential of CD133⁺CD45⁻Ter119⁻ cells, the present inventors attempted to conduct clone-sorting of the cells by flow cytometry, and then to culture the cells in the individual wells of a 96-well plate coated with type IV collagen. As with the semi-clonal low-density culture conditions, the cells isolated from a CD133⁺CD45⁻Ter119⁻ cell subpopulation could form small colonies or large colonies, depending on whether or not the liver has been pretreated with DDC, in each well (FIG. 7). Clonal colony formation in single cell culture of CD133⁺CD45⁻Ter119⁻ cells allows investigations and characterization of the cells that have differentiated from the original cell. An immunofluorescence staining analysis using antibodies against Alb and CK7 revealed that after 6 days and 18 days of cultivation, the majority of the small colonies derived from the livers of DDC mice and the livers of No-DDC mice are configured with cells expressing only CK7 (FIGS. 8A, B). By contrast, after 6 days of cultivation, half of the large colonies were configured with CK7⁺ cells, Alb⁺CK7⁺ cells, and a few Alb⁺ cells, whereas the remaining half of the colonies were configured exclusively with CK7⁺ cells (FIGS. 8A, B). Finally, 18 days later, however, more than 70% of the large colonies were configured with Alb⁺ cells, CK7⁺ cells, and a few Alb⁺CK7⁺ cells (FIGS. 8A, B). Irrespective of colony size and the duration of cultivation, no colonies formed by Alb⁺ cells only were observed (FIG. 8B). Analysis by RT-PCR also showed that the cells contained in the large colonies expressed multiple genes that encode markers of hepatocytes and cholangiocytes (including genes that are detected in mature hepatocytes, such as tryptophan-2,3-dioxygenase, glutathione S-transferase, and glutamine synthase) (FIG. 9). It is interesting to note that genes that have been reported as oval cell markers, such as cKit, CD34 and Thy1, are not expressed in all large colonies. Furthermore, in large colonies cultured for 3 weeks or more, many binuclear Alb⁺ cells with sufficiently accumulated glycogen that are similar to functionally mature hepatocytes appeared in the vicinity of the center of each colony (FIGS. 8C, D). Because the CD133⁺CD45⁻Ter119⁻ cells isolated from the livers of DDC mice are initially positive for CK7, but negative for Alb (FIG. 5B), cells capable of forming large colonies were thought to give rise to progeny that differentiate into Alb⁺ mature hepatocytes during the culturing process.

### Example 4

### Self-Renewing Capacity of CD133⁺CD45⁻Ter119⁻ Cells

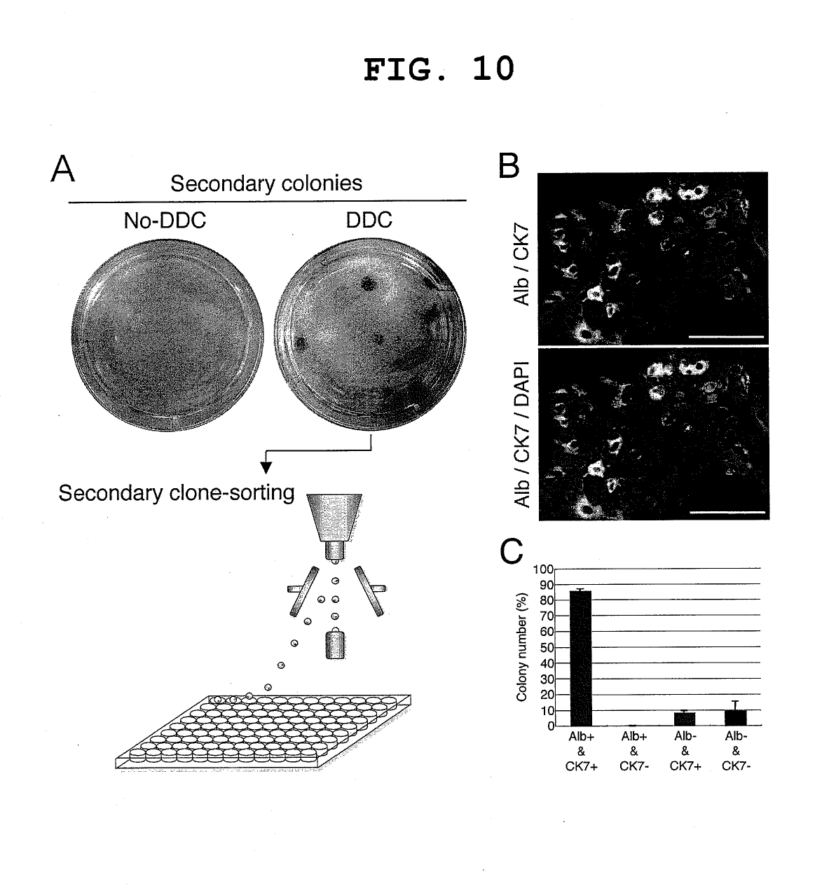

In order to examine the CD133⁺CD45⁻Ter119⁻ cells isolated from the liver of a DDC mouse for self-renewing capacity, a subcloning experiment was performed. The cells in each clonal colonies formed in each well of a 96-well plate coated with type IV collagen were trypsinized, and re-sown to a 6-well plate coated with type IV collagen. After 14 days of cultivation, many large colonies (5 to 10 colonies from a single colony) emerged from subcultured cells originally derived from the liver of a DDC mouse. When the liver of a No-DDC mouse was used, however, no large colonies were produced (FIG. 10A). The cells in these secondary colonies were trypsinized and subjected to clone sorting, and single cell culture was performed. Although the ratio of re-sorted cells capable of forming large colonies was low (1 to 3%), more than 80% of the colonies formed contained cells differentiated into both Alb⁺ cells and CK7⁺ cells by day 18 of cultivation (FIG. 10C). As with the initially sorted cells that formed large colonies, the large colonies formed again by the re-sorted cells began to produce Alb⁺ cells gradually during the cultivation, but no colonies containing Alb⁺ cells only were formed (FIG. 10C). These data show that the CD133⁺CD45⁻Ter119⁻ cells that form large colonies, isolated from the liver of a mouse fed on DDC, not only give rise to Alb⁺ hepatocytes and CK7⁺ cholangiocytes as descendants thereof, but also continue to maintain cells capable of differentiating into hepatocytes and cholangiocytes by self-renewing cell division throughout the cultivation.

### Example 5

### Differentiating Capacity and Hepatic Tissue Reconstitution Capacity of CD133⁺CD45⁻Ter119⁻ Cells In Vivo

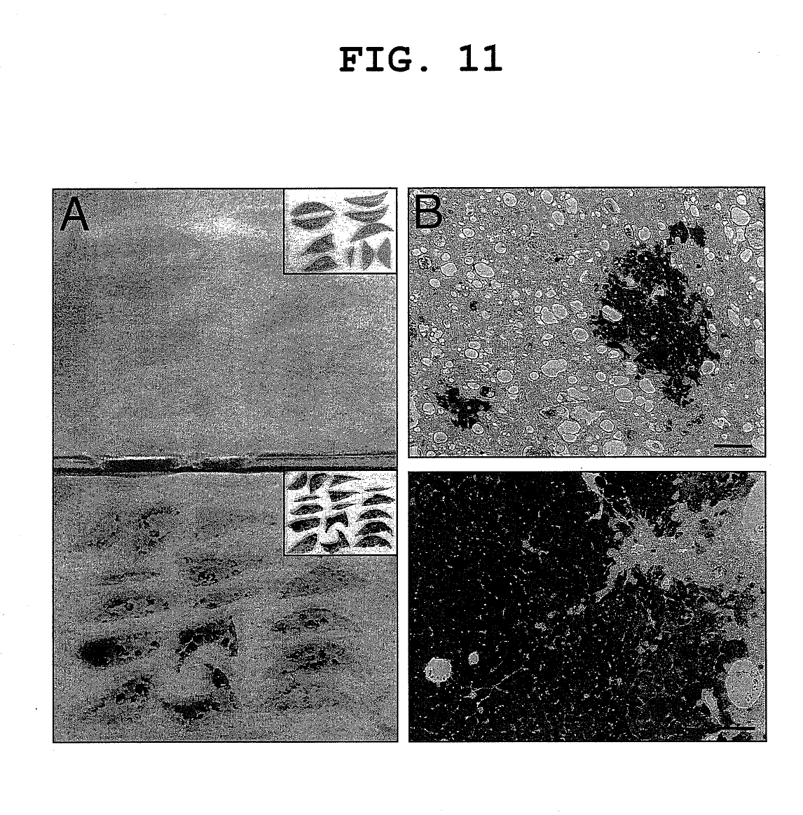

Reconstitution and functional repair of injured tissue are one of the special roles of organ stem cells and organ progenitor cells. As reported previously, the oval cells isolated from the liver of an adult mouse fed on DDC have the liver reconstitution capacity by being transplanted to the liver of a FAH-deficient mouse (reference 21). To determine whether or not the CD133⁺CD45⁻Ter119⁻ cells isolated from hepatic cells of a mouse fed on DDC are also capable of likewise hepatic tissue reconstitution and can be defined as oval cells, these cells were transplanted intrasplenically to the livers of FAH−/− mice. The FAH−/− mice used here were prepared from ES cells with targeted-disruption of FAH (see the “Generation of FAH mutant mouse” section above, FIG. 2), and are indistinguishable from other FAH mutant mice in terms of phenotype. In the present invention, the present inventors used cloned progeny cells derived from a single CD133⁺CD45⁻Ter119⁻ cell as donor cells to exclude the possibility of contamination with other cell types capable of reconstituting hepatic tissue (i.e., mature hepatocytes). After elapse of 1 and 2 months following the transplantation, the FAH⁺ donor cells engrafted diffusively and reconstituted hepatic nodules in all recipient mice. These mice were able to survive long-term discontinuation of 2-(2-nitro-4-trifluoromethylbenzoyl)-1,3-cyclohexanedione (NTBC) (FIG. 11A-C). Furthermore, immunofluorescence staining and periodic acid Schiff reagent (PAS reagent) staining demonstrated that the FAH⁺ donor-derived cells expressed Alb and sufficiently accumulated glycogen (data not shown). These data show that because there is no possibility of contamination of other lineage cells in the pool of the donor cells, the proliferating CD133⁺CD45⁻Ter119⁻ cells that were isolated from the liver of a mouse fed on DDC are capable of reconstituting hepatic tissues by producing morphologically and functionally mature hepatocytes. Hence, the proliferating CD133⁺CD45⁻Ter119⁻ cells that were isolated from the liver of a mouse fed on DDC can be defined as hepatic oval cells.
Reference 21: Wang X, Foster M, Al-Dhalimy M, et al. The origin and liver repopulating capacity of murine oval cells. Proc Natl Acad Sci USA 2003; 100 Suppl 1: 11881-11888.

### Example 6

### Hepatocarcinoma Formation by p53-Deficient CD133⁺CD45⁻Ter119⁻ Cells

As in the experimental system in which DDC is fed to mice, in some experimental models of hepatocarcinogenesis in rodents using Dipin (cisplatin) or the choline-deficient plus ethionine diet, and also in human liver lesions that develop more frequently to hepatocarcinoma (hepatitis C virus, hemochromatosis, alcoholic liver disease and the like), oval cells have been observed (references 1 to 5).

This suggests that the transformed oval cells may possibly be a candidate for the origin of hepatocarcinoma in both rodents and humans. This idea is supported by recently obtained evidence for the presence of putative cancer-initiating cells or cancer stem cells in many types of cancer (references 22 to 30). Agreeing with this idea, it is reported that oval cells derived from p53-deficient mice fed on choline-deficient plus ethionine diet gave rise to an immortalized cell lines and generate tumor cells after injection into nude mice (reference 31).

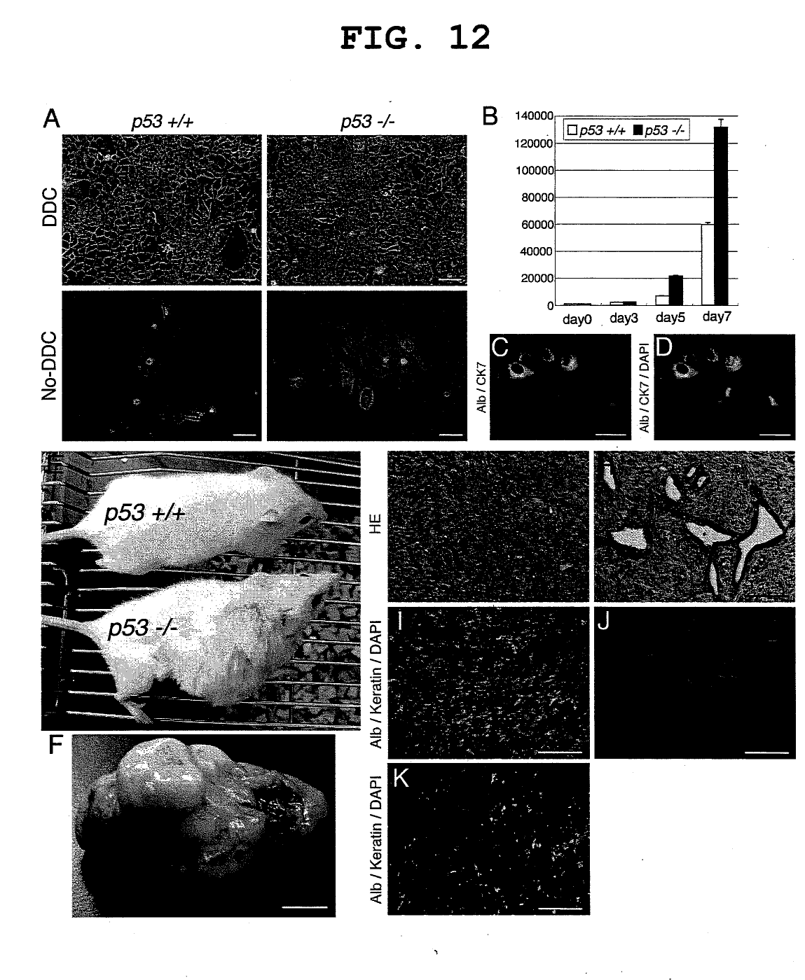

In order to determine whether or not CD133⁺CD45⁻Ter119⁻ cells are capable of initiating tumorigenesis, CD133⁺CD45⁻Ter119⁻ cells were isolated from the liver of a p53−/− mouse fed on DDC. The CD133⁺CD45⁻Ter119⁻ cells isolated from the liver of a p53−/− mouse fed on DDC formed large colonies and proliferated, whereas the cells isolated from the liver of a p53−/− mouse not fed on DDC did not form a large colony or proliferated. This finding was similar to the corresponding finding in the CD133⁺CD45⁻Ter119⁻ cells derived from the liver of a wild type mouse (FIG. 12A). However, although the CD133⁺CD45⁻Ter119⁻ cells obtained from the liver of a p53−/− mouse fed on DDC were not influenced in terms of their capacity of differentiating into hepatocytes and cholangiocytes (FIG. 12C-D), they had smaller sizes and remarkably higher proliferative capacity, compared with those derived from the liver of a wild type mouse (FIG. 12A-B). Clonogenic cells expanded from CD133⁺CD45⁻Ter119⁻ cells derived from the liver of a p53−/− mouse fed on DDC were injected subcutaneously into non-obese diabetic/severe combined immunodeficient (NOD/SCID) mice; 2 months later, a tumor containing Alb⁺ hepatocytes and keratin⁺ cholangiocytes, which form a bile duct-like structure, was formed in all animals. On the other hand, no tumors were formed in the liver having cells derived from a wild type mouse transplanted thereto (FIG. 12E-K). Hence, the CD133⁺CD45⁻Ter119⁻ cells isolated from the liver of a p53−/− mouse fed on DDC are capable of forming a tumor having the features of both hepatocellular carcinoma and cholangiocarcinoma; these cells can be characterized as hepatic oval cells that acquire the capability of inducing a tumor by deleting the function of p53.
Reference 22: Bonnet D, Dick J E. Human acute myeloid leukemia is organized as a hierarchy that originates from a primitive hematopoietic cell. Nat Med 1997; 3: 730-737.
Reference 23: Al-Hajj M, Wicha M S, Benito-Hernandez A, et al. Prospective identification of tumorigenic breast cancer cells. Proc Natl Acad Sci USA 2003; 100: 3983-3988.
Reference 24: Singh S K, Hawkins C, Clarke I D, et al. Identification of human brain tumour initiating cells. Nature 2004; 432: 396-401.
Reference 25: Collins A T, Berry P A, Hyde C, et al. Prospective identification of tumorigenic prostate cancer stem cells. Cancer Res 2005; 65: 10946-10951.
Reference 26: Ricci-Vitiani L, Lombardi D G, Pilozzi E, et al. Identification and expansion of human colon-cancer-initiating cells. Nature 2007; 445, 111-115.
Reference 27: O'Brien C A, Pollett A, Gallinger S, et al. A human colon cancer cell capable of initiating tumour growth in immunodeficient mice. Nature 2007; 445: 106-110.
Reference 28: Prince M E, Sivanandan R, Kaczorowski A, et al. Identification of a subpopulation of cells with cancer stem cell properties in head and neck squamous cell carcinoma. Proc Natl Acad Sci USA 2007; 104: 973-978.
Reference 29: Zucchi I, Sanzone S, Astigiano S, et al. The properties of a mammary gland cancer stem cell. Proc Natl Acad Sci USA 2007; 104: 10476-10481.
Reference 30: Li C, Heidt D G, Dalerba P, et al. Identification of pancreatic cancer stem cells. Cancer Res 2007; 67: 1030-1037.
Reference 31: Dumble M L, Croager E J, Yeoh G C, et al. Generation and characterization of p53 null transformed hepatic progenitor cells: oval cells give rise to hepatocellular carcinoma. Carcinogenesis 2002; 23: 435-445.

This application is based on patent application No. 39015/2008 filed in Japan, the contents of which are hereby incorporated by reference.

### [Sequence Listing Free Text][0043]

SEQ ID NO: 1: CK7 RT-PCR primer
SEQ ID NO: 2: CK7 RT-PCR primer
SEQ ID NO: 3: primer for amplification of 5′ arm
of targeting vector sequence
SEQ ID NO: 4: primer for amplification of 5′-arm of
targeting vector sequence
SEQ ID NO: 5: primer for amplification of 3′-arm of
targeting vector sequence
SEQ ID NO: 6: primer for amplification of 3′-arm
of targeting vector sequence
SEQ ID NO: 7: primer, FAHwt-F
SEQ ID NO: 8: primer, FAHmut-F
SEQ ID NO: 9: primer, FAHwt/mut-R

1. A method of separating and/or acquiring hepatic oval cells from a mammal, comprising a step for examining the expression of CD133, CD45 and Ter119.

2. The method of claim 1, further comprising a step for sorting cells that express CD133 and do not express CD45 or Ter119.

3. The method of claim 1, further comprising a step for inducing the production of hepatic oval cells.

4. The method of claim 1, further comprising a step for culturing hepatic oval cells in the presence of a growth factor and an extracellular matrix.

5. The method of claim 4, wherein the growth factor is HGF and/or EGF.

6. The method of claim 4, wherein the extracellular matrix is collagen or laminin.

7. Hepatic oval cells separated and/or acquired from the liver of a mammal by the method of claim 1.

8. Hepatic oval cells that exhibit the pattern of CD133⁺, CD45⁻ and Ter119⁻ for the three cell surface markers CD133, CD45 and Ter119.

9. A method for screening for a substance that influences differentiation in the liver of a mammal, comprising the following steps;
(1) a step for reacting hepatic oval cells and a test substance,
(2) a step for measuring the expression of a liver marker in the cells after the reaction.
